(19) United States
(12) Patent Application Publication
(10) Pub. No.: US 2006/0025408 A1
(43) Pub. Date: Feb. 2, 2006

(54) Process for the preparation of crystalline form-1 of 4-hydroxy-2-methyl-N-(5-methyl-2-thiazolyl)-2H-1,2-benzothiazine-3-carboxamide-1,1-dioxide

(76) Inventors: Sundaram Venkataraman, Hyderabad (IN); Muppa Kishore Kumar, Hyderabad (IN); Koilkonda Purandhar, Hyderabad (IN); Bairy Kondal Reddy, Medak (IN); Lekkala Amarnath Reddy, Hyderabad (IN); Golla Chinamala Kondaiah, Hyderabad (IN); Keshaboina Sreenath, Warangal (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 10/909,925

(22) Filed: Aug. 2, 2004

(51) Int. Cl. C07D 279/16; A61K 31/5415

(52) U.S. Cl. 514/226.5; 544/49

(57) ABSTRACT

The present invention is related to an improved process for preparing crystalline Form I of meloxicam, which produces the Form I in high yield.

The present invention relates to the improved process for the preparation of crystalline Form-1 of Meloxicam. Meloxicam is chemically known as 4-Hydroxy-2-methyl-N-(5-methyl-2-thiazolyl)-2H-1,2-benzothiazine-3-carboxamide-1,1-dioxide, which is represented by the following Formula (1).

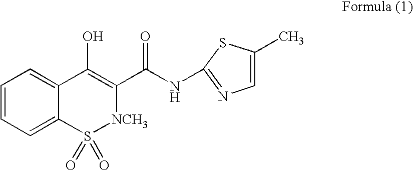

U.S. 2003/0109701 describes a process for the preparation of Crystalline Form-1 of Meloxicam and other novel crystalline forms. According to the said patent, crystalline Form I of Meloxicam was prepared by a process, which involves dissolving Meloxicam in a mixture consisting of water and NaOH, and subsequent addition of an acid for precipitation of the crystalline Form I of Meloxicam. The said patent also describes the process for the preparation of crystalline Form I of Meloxicam, which involves dissolving Meloxicam in a mixture consisting of water, NaOH and an organic solvents like alcohols, xylene, toluene and dimethylformamide (DMF), at a reflux temperature followed by addition of an acid to get a pH of 3 to 5.5 further cooling and isolating the precipitate to get the crystalline Form I of Meloxicam.

The present invention relates to an improved process for the preparation of Meloxicam Form I, which involves the crystallization of said compound in acetone under inbuilt pressure vessel afford crystalline Form 1 in good yields with excellent purity.

In another aspect the present invention provides a method that has edge over the prior art methods as it is avoiding enormous effluent. It is also simple, eco-friendly and well suited for large-scale production.

According to one embodiment of the present invention, crystalline Form I of meloxicam can be made by dissolving 4-hydroxy-2-methyl-n-(5-methyl-2-thiazolyl)2h-1, 2-benzothiazine-3-carboxamide 1,1-dioxide (Formula 1 A) in acetone; adding activated carbon in the solution; heating the solution with activated carbon upto about 85 to 90° C. for about 1 to 2 hours with agitation; removing activated carbon; cooling the filtrate to about 0 to 5° C. with stirring for about hours to give precipitates; and isolating the precipitates to give Form I of meloxicam.

The removal of the activated carbon is done preferably using a hyflow bed with a pressure of about 2 to 3 kg/cm² at about 40 to 45° C., and the precipitates can be isolated by conventional methods, such as filtration. When isolated, the precipitates can be washed with acetone without risk losing its structural characteristics.

Meloxicam itself can be prepared from sodium saccharine dihydrate (II) by a chemical route shown in Scheme 1, wherein Sodium saccharine dihydrate is converted to saccharine acetic acid ester (III), which converted in sequence to methyl-4-hydroxy-2-methyl-2H-1,2-benzothiazine-3-carboxylate 1,1-dioxid and then to 4-hydroxy-2-methyl-N-(5-methyl-2-thiazoyl)-2H-1,2-benzothiazine-3-carboxamide-1,1-dioxide.

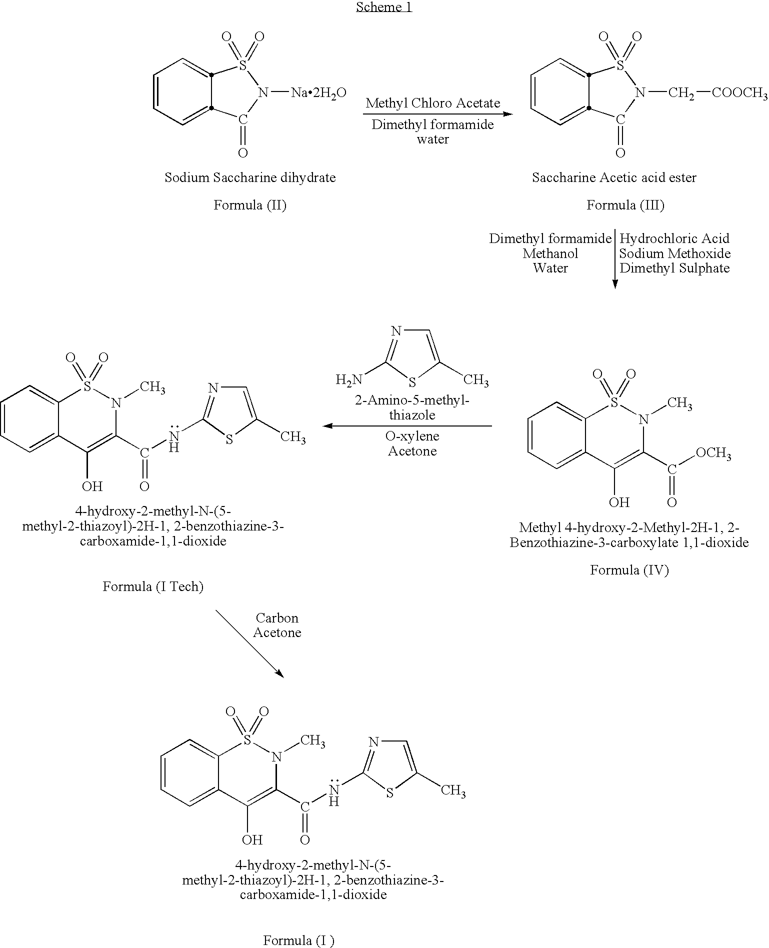

Saccharine acetic acid ester (III) can be prepared by dissolving saccharine dihydrate and methyl chloroacetate in dimethylformamide; heating the solution) to about 115 to 120° C. for about 1 to 2 hours; when it is cooled to about 25-30° C., adding water to the heated solution, which was then stirred for about 30 to 45 minutes; and isolating precipitated solids.

The dimethylformamide solution does not have to be a clear solution prior to the heating. The solids can be isolated by any conventional method such as filtration, in which case the filtered solids can be washed with water and dried for about 30 to 45 minutes before being used for the next step. Saccharine acetic acid ester (III) is readily converted to methyl-4-hydroxyl-2-methyl-2H-1,2-benzothiazine-3-carboxalate-1,1-dioxide (IV), which is prepared by mixing saccharine acid ester of Formula III in dimethylformamide with sodium methoxide in dimethylformamide under inert atmosphere at about 15 to 25° C.; adding slowly dimethyl sulphate into the resulting solution over about 1 to 1.5 hours; adding water to the reaction solution upon confirmation of the reaction completion; adjusting pH of resulting aqueous mixture to about between 2 and 4 to get precipitation; isolating the precipitates, which washed with water and then with methanol; and drying the washed precipitates under reduced pressure to get methyl-4-hydroxyl-2-methyl-2H-1,2-benzothiazine-3-carboxalate-1,1-dioxide (IV).

The mixing of the ester compound and sodium methoxide is preferably done slowly. The isolation of the precipitates should be done once the TLC test show the reaction is completed in order to achieve the maximum yield and can be made by conventional methods such as filtration. The washing of the precipitates with methanol may be performed by adding the precipitates in methanol at about 25-35° C., stirring the mixture and filtering the precipitates again.

Meloxicam, 4-hydroxy-2-methyl-N-(5-methyl-2-thiazolyl)₂H-1,2-benzothiazine-3-carboxamine-1,1-dioxide can be prepared by adding methyl-4-hydroxyl-2-methyl-2H-1,2-benzothiazine-3-carboxalate-1,1-dioxide (IV) and 2-amino-5-methyl-thiazole to xylene; refulxing the resulting reaction mass for about 8-9 hours during which about 2.5-3.5% of the solvent was distilled off from the reaction mass for every one hour; adding ortho xylene to the refluxed reaction mass at 130-135° C. to get first xylene mixture; refluxing the first xylene mixture for about 8-9 hours with removal of the distillated solvent (about 2.5-3.5% of the solvent) for every one hour; adding again ortho xylene to the refluxed first xylene mixture at 130-135° C. to get second xylene mixture; refluxing the second xylene mixture for about 8-9 hours with removal of the distillated solvent (about 2.5-3.5% of the solvent) for every one hour; cooling the second xylene mixture to about 25-35° C. and stirring it for 1-2 hours; filtering the cooled second xylene mixture to get technical grade meloxicam.

The adding-xylene-refluxing step may be repeated more than two times. The filtered meloxicam can be dried under pressure, and optionally can be purified by recrystallization in dichloromethane followed by slurring in acetone;

The following example is only illustrative and is not intended to limit the scope of the invention in any way.

### EXAMPLE 1

### PREPARATION OF SACCHARINE ACID ESTER (FORMULA III)

Dimethylformamide (52 L), 50 kg of sodium saccharine and 28.50 kg of methyl chloro acetate were added into a reactor, which was then heated to 115 to 120° C., and the reaction mass was maintained for about 1½ to 2 hours at the same temperature. The reaction mass was cooled to 25 to 35° C., added with 95 liters of water, and then stirred at 25 to 35° C. for about 30 to 45 minutes. The reaction mass was centrifuged, and the obtained cake was washed with 100 liters of water. The washed cake was spin-dried by spin drying for about 30 to 45 minutes. Then the wet solid was dried at 75-80° C. for about 3 hours to get 52 kg of the desired compound (98%).

### EXAMPLE 2

### PREPARATION OF METHYL 4-HYDROXY-2-METHYL-2H-1,2-BENZOTHIAZINE-3-CARBOXALATE 1,1-DIOXIDE (IV)

Dimethylformamide (350 L) was added into a reactor under nitrogen atmosphere, and 42.5 kg of sodium methoxide was added to the reactor. The resulting reaction mass was cooled to 15 to 25° C., and a solution of 50 kg of saccharine acid ester (III) in 150 liters dimethylformamide was added to the cooled reaction mass at the same temperature over 30 to 45 minutes. Then, dimethylsulphate (74.5 kg) was added to the reaction mass over about 1-1½ hour. After the resulting reaction mixture was maintained at 15 to 30° C. for about 30 to 45 minutes, 920 liters of water was added to the reaction mixture. The temperature of the aqueous mixture was maintained at about 15 to 25° C. and 25 liters of hydrochloric acid was added to the aqueous mixture to adjust its pH to between about 2 to 4. After the acidified mixture was stirred for about 30 to 45 minutes, the mixture was filtered. The filtered wet solid was washed with 240 liters of water and then spin-dried. The spin-dried wet solid was added to 125 liters of methanol, which was stirred for about 30 to 45 minutes at 25 to 35° C. The methanol mixture was filtered to isolate the solid, which was washed with 100 liters of methanol thoroughly and spin dried for 30 to 45 minutes under reduced pressure. Further the solid was dried at 55 to 60° C. under reduced pressure for about 3 hours to get the titled compound. (Yield: 35-42 kg)

### EXAMPLE 3

### PREPARATION OF (4-HYDROXY-2-METHYL-N-(5-METHYL-2-THIAZOLYL)₂H-1,2-BENZOTHIAZINE-3-CARBOXAMIDE 1,1-DIOXIDE (I, Technical grade)

Ortho xylene (1800 L), 18 kg of methyl 4-hydroxy-2-methyl-2H-1,2-benzothiazine-3-carboxalate1,1-dioxide (IV) and 8.6 kg of 2-amino-5-methyl-thiazole were added into a reactor. The reaction mass was heated to 142 to 145° C. and maintained under reflux maintained for 8 to 9 hours at the same temperature. During the reflux, 50-55 L of distillate was collected separately for every 1 hour. The reaction mass was cooled to 130 to 135° c., and 400 liters of ortho xylene was added to the reaction mass, which was then heated to 142 to 145° C. and maintained for 8 to 9 hours at the same temperature. During the reflux, 50-55 L of distillate was collected separately for every 1 hour. The reaction mass was cooled to 130 to 135° c., and 400 liters of ortho xylene was added to the reaction mass, which was then heated to 142 to 145° C. and maintained for 8 to 9 hours at the same temperature. Similarly, During the reflux, 50-55 L of distillate was collected separately for every 1 hour. The reaction mass was cooled to 130 to 135° c., and 400 liters of ortho xylene was added to the reaction mass, which was then heated to 142 to 145° C. and maintained for 8 to 9 hours at the same temperature. During the reflux, 50-55 L of distillate was collected separately for every 1 hour. The reaction mass was then cooled to 25 to 35° C., and stirred for about 1½-2 hours at the same temperature. The reaction mass was filtered and the filtered wet solid was washed with acetone thoroughly. The wet solid was suck dried for 45 to 60 minutes to obtain a technical grade titled compound. (Yield: 16.0 kg, 88.8%)

### EXAMPLE 4

### PURIFICATION OF 4-HYDROXY-2-METHYL-N-(5-METHYL-2-THIAZOLYL)₂H-1,2-BENZOTHIAZINE-3-CARBOXAMIDE 1,1-DIOXIDE (I)

Acetone (1530 L) was added into a reactor along with 4-hydroxy-2-methyl-N-(5-methyl-2-thiazolyl) 2H-1,2-benzothiazine-3-carboxamide 1,1-dioxide (18 kg, technical grade) and carbon (1 kg). The reaction mass was heated to 85 to 90° C. and maintained for 1 to 1½ hours at same temperature. The reaction mass was filtered at 45 to 55° C. through a leaf filter containing hyflow bed under pressure at 2.0 to 3.0 kg/cm². The filtrate was cooled to 45-55° C., and acetone (180 L) was added to the cooled filtrate, which was then heated to 80-90° C. by applying hot water, and the aqueous mixture was maintained at the same temperature for 5-10 minutes. The aqueous mixture was then cooled slowly to 0-5° C. over 2.5-3 hours and maintained at the same temperature for 3-4 hours. Then the aqueous mixture was centrifuged, and the cake was washed with acetone (35 L). The washed solid was then spin-dried for 30 to 45 minutes. The solid was further dried at 85 to 95° C. under reduced pressure to get the crystalline Form I of Meloxicam (16.0 kg, 88.8%).

We claim:

1. An improved process for the preparation of crystalline Form I of Meloxicam, comprising
a. adding carbon to the mixture of 4-hydroxy-2-methyl-n-(5-methyl-2-thiazolyl)2h-1,2-benzothiazine-3-carboxamide 1,1-dioxide and acetone;
b. heating the mixture of step (a) to 65-110° C.;
c. removing carbon at about 40-55° C. under pressure by maintaining gauge pressure at about 1.0 to 5.0 kg/cm² through hyflow bed;
d. cooling the filtrate of step (c) slowly to 0-5° C.; and
e. isolating the separated solid of step (d).

2. The process according to claim 1, wherein the cooling is taken place over about 2-3 hours.

3. The process according to claim 1, wherein the pressure gauge is maintained at 2.0 to 3.0 kg/cm².
